(19) United States
(12) Patent Application Publication
(10) Pub. No.: US 2010/0196493 A1
(43) Pub. Date: Aug. 5, 2010

(54) METHOD FOR PRODUCING STABLE POWDER COMPOSITIONS

(75) Inventor: Pierre Buisson, Lagord (FR)

Correspondence Address: YOUNG & THOMPSON, 209 Madison Street, Suite 500, Alexandria, VA, 22314, US

(73) Assignee: INNOV'IA, La Rochelle (FR)

(21) Appl. No.: 12/669,868

(22) PCT Filed: Jul. 18, 2008
(86) PCT No.: PCT/FR08/01069

§ 371 Date: Apr. 22, 2010

(30) Foreign Application Priority Data
Jul. 20, 2007 (FR) 0705307

(51) Int. Cl. A61K 9/14; A61K 38/54; A61K 38/43; A61K 35/74; A61K 31/122; A61P 43/00; A23L 1/00; A23K 1/00; A23L 1/302; A23L 1/28; B05D 7/00

(52) U.S. Cl. 424/490; 424/94.2; 424/93.45; 514/691; 426/303; 426/96; 426/72; 426/62; 427/212

(57) ABSTRACT

The invention relates to the use of at least one viscous compound, the viscosity of which is greater than 5 Pa·s⁻¹, to substantially prevent the degradation of a substance sensitive to temperatures of 20° C. to 100° C. and/or pressures of 10⁶ to 10⁷ Pa and/or a relative humidity of 60% to 100%, contained in a powder composition, the powder composition containing particles including: a core containing the sensitive substance and at least one viscous compound as impregnating agent for the sensitive substance and a coating for the core, which contains at least one viscous compound as sticking agent.

The invention relates to a novel method for preparing stable powder compositions. In particular, the invention relates to a novel method for preparing powder compositions comprising a substance sensitive in particular to temperature and/or pressure and/or relative humidity.

Sensitive substances are often active substances used in pharmacology or in compositions used in different fields such as human or animal food, detergents, etc. Proteins, in particular enzymes, vitamins, bacteria, yeasts, antioxidants, carotenoids, essential oils or also pharmacologically active substances such as antibiotics, can be mentioned in particular as examples.

However, during the preparation of these compositions and in particular during the granulation operations, the substances are subjected to several stresses. In fact, the main characteristic of the machines performing these operations is that they cause the materials constituting the final composition to pass through a die having holes of different sizes, thus creating significant shear, leading to a rise in temperature in the core of the material. Moreover, in order to make this passage easier, the material is pre-heated in a standard fashion and steam is also added to bring about cohesion. These different temperature, pressure and humidity stresses can be harmful to the sensitive substances, which can then lose some of their activity during these operations.

Different solutions have been proposed in the past to solve these problems.

For instance, the patent EP 0 569 468 proposes a method for preparing granules containing (an) enzyme(s) intended for animal food and coated with a fat or wax having a high melting point, in order to increase resistance to the granulation conditions. However, such a coating has the disadvantage that the granules take a long time to dissolve, which reduces the bioavailability of the enzymes for the animal.

Similarly, the application WO 00/47060 describes a method for preparing granules containing (an) enzyme(s) coated with polyethylene glycol. This coating then allows the stability of the enzymes during a granulation stage to be increased but only in the presence of additives such as water-soluble inorganic salts (for example ZnSO₄) or also trehalose. These additives are not necessarily of interest for the use of these granules under consideration and only serve to increase the cost.

A method for preparing granules containing (an) enzyme(s) coated with a polyolefin was also developed in the application WO 03/059087, in particular for use in the field of animal food. In particular, the coating used then allows the stability of the enzyme(s) during the granulation stages to be increased. However, this requires the use of polymer derivatives which are not necessary for the animal food and which increase the manufacturing cost of the granules.

The application WO2005/074707 describes stabilized phosphatase formulations, the stabilizing agent being chosen exclusively from agar, algin, carrageenan, furcelleran, ghatti gum, tragacanth gum, gum karaya, guaran, carob bean gum, tamarind seed gum, arabinogalactan and xanthan gum. These formulations can be coated with polymers chosen in particular from cellulose derivatives having a molecular weight comprised between 6000 and 80000. This application makes no mention of the viscosity of the products used.

The application EP0600775 describes active agents stabilized by coating. The coating composition comprises a film-forming agent which can be in particular a cellulose derivative such as cellulose acetate, ethyl cellulose or methyl cellulose and a pore-forming agent which allows a rapid breakdown of said coating composition by forming preferential access points for the biological fluids.

The application WO03/059086 describes a granule formulation and its preparation method. However, no information on the viscosity of the products used is given.

One aspect of the invention is therefore to propose a novel method for preparing powder compositions containing at least one sensitive substance which is able to withstand high temperature, pressure and relative humidity conditions and which is stable during transport and storage, this method being easy to implement and inexpensive.

A subject of the present invention is more particularly the use of at least one viscous compound, the viscosity of which is greater than 5 Pa·s⁻¹, to substantially prevent the degradation of a substance sensitive to temperatures of 20° C. to 100° C. and/or pressures of 10⁶ to 10⁷ Pa and/or a relative humidity of 60% to 100%, contained in a powder composition, said powder composition containing particles comprising:
a core containing said sensitive substance and at least one viscous compound as impregnating agent for the sensitive substance and
a coating for the core, which contains at least one viscous compound as sticking agent.

The viscous compounds used in the present invention have the significance of being practically hydrophobic when subjected to high pressures of the order of 10⁶ to 10⁷ Pa, but recover all their solubility properties when put back into solution. This thus makes it possible to protect the sensitive substances during the granule preparation by a granulation stage while later guaranteeing a good bioavailability of the sensitive substances as a result of the short dissolution time of the particles of the powder composition. In particular, when the powder compositions are intended for animal food, the sensitive substance, which is also the active ingredient, is released directly into the animal's digestive tract.

By “granulation” is meant a stage allowing the preparation of granules in the form of cylinders 1 to 10 mm in diameter and 1 to 5 cm in length by passing them through a die. This stage is characterized by high temperature and pressure stresses and by shear stress, as well as by the presence of steam, these stresses being able to damage the sensitive substances.

The viscosity measurements are carried out using a BROOKFIELD viscometer, model LVDV-E fitted with a coaxial cylinder system, using spindle no. 18 or 31 depending on the viscosity, in a measurement chamber with a temperature controlled using a thermostated bath. These measurements are carried out at 20° C. for aqueous solutions with 10% dry extract of the viscous compound, at different rotational speeds of the spindle of 10 to 100 rpm. The retained viscosity values are the values obtained at the highest rotational speeds, integrating the limit values of the torsion torque (maximum 80%) of the device.

The viscous compound is advantageously chosen from vegetable gums or fermentation products, starchy bases, chitins or cellulose derivatives, such as carboxymethyl cellulose (CMC), methyl cellulose, ethyl cellulose, propyl cellulose, hydroxymethyl cellulose, hydroxyethyl cellulose, hydroxypropyl cellulose, hydroxypropyl methyl cellulose, ethyl hydroxyethyl cellulose, or microcrystalline cellulose.

In a particularly advantageous embodiment of the present invention, the viscous compound is carboxymethyl cellulose (CMC). This compound provides protection for the sensitive substance under conditions of heat and pressure, which makes it practically insoluble whereas, put back into solution, it hydrates and becomes bioavailable again.

By “sensitive substance” is meant any substance which is liable to lose some or all of its activity when subjected to high temperatures and/or pressures and/or relative humidity. These stresses may be encountered in particular during the preparation of compositions containing said substance, and more particularly during the granulation stages. This substance can be in particular a mixture of sensitive substances.

By “degradation” is meant any conversion of said sensitive substance which involves a loss of activity, partial or total, whether by denaturation of said substance or by changing the proportions in a composition comprising several substances, some of them in particular being volatile (for example in the case of an essential oil).

The sensitive substance can be chosen in particular from proteins, in particular enzymes, vitamins, bacteria, yeasts, antioxidants, carotenoids, essential oils or pharmacologically active substances such as antibiotics.

Mention can be made, by way of example, of superoxy dismutase for the enzymes, riboflavin or ascorbic acid for the vitamins, lactobacillus for the bacteria, saccharomyces cerevisiae for the yeasts, polyphenols or vitamin E for the antioxidants, lutein, astaxanthin, lycopene, xanthophyll or carotenes for the carotenoids, oils obtained from garlic, thyme or rosemary for the natural essential oils and amoxicillin or tylosin for the antibiotics.

This sensitive substance can be used in particular in the field of animal or human food, in the pharmaceutical or veterinary field, in the field of detergents, of cleaning and washing products, in particular for clothing.

By “powder composition” is meant powders comprising particles having an average grain size of 30 to 3 000 μm on average.

The particles of the powder composition include two very distinct parts: a core and a coating for this core which forms a protective film. The core is constituted by the sensitive substance and at least one viscous compound which acts as impregnating agent. As for the coating, it contains at least one viscous compound which acts as sticking agent. The viscous compound is therefore involved at two levels, since it is present in both the core and the coating. It is possible to use two different viscous compounds, one contained in the core and the other in the coating.

The viscous compound used as impregnating agent makes it possible to ensure a homogeneous mixture without demixing of the elements constituting the composition, while binding all of the grains together.

The viscous compound used as sticking agent, in turn, makes it possible to achieve permanent protection. Thus, during a granulation stage, it ensures on the one hand a protection against the high humidity and temperature conditions encountered when the composition is being brought up to temperature using steam during mixture with the foodstuff and, on the other hand, a protection against the high pressure conditions and shearing conditions encountered when passing through the die.

The core of the particles of said powder composition advantageously contains at least one support of said sensitive substance.

In fact, due to its absorbent nature, the support is a material chosen as already being in the dry state. It protects the sensitive substance by stabilizing the activity of the water when the support is in contact with the sensitive substance in liquid form, sprayed during drying phases.

This support can be in particular starch, flours, in particular wheat, corn, manioc or rice flour, talc, beet pulp, maltodextrin, salts such as calcium carbonate or corn distiller's grain.

Advantageously, the impregnating agent and the sticking agent are identical.

However, the viscous compound has very different functions depending on whether it is used as impregnating agent or as sticking agent, as already mentioned above, even if it is the same compound.

In a particularly advantageous embodiment, the impregnating agent and the sticking agent contain or are constituted by carboxymethyl cellulose (CMC).

In a particular embodiment, the sensitive substance represents 5 to 40%, preferably 10 to 30%, dry extract of the powder composition, the support represents 10 to 60%, preferably 20 to 50%, dry extract of the powder composition, the impregnating agent represents 5 to 40%, preferably 10 to 30%, dry extract of the powder composition and the sticking agent represents 5 to 30%, preferably 10 to 20%, dry extract of the powder composition.

In another particular embodiment, the sensitive substance is constituted by a mixture of enzymes constituted principally by β-glucanase, xylanase and cellulase, the support is constituted by wheat flour, the impregnating agent and the sticking agent are constituted by carboxymethyl cellulose (CMC).

The present invention also relates to a stable powder composition containing particles comprising:
a core containing at least one substance sensitive to temperatures of 20° C. to 100° C. and/or pressures of 10⁶ to 10⁷ Pa and/or a relative humidity of 60% to 100% and at least one viscous compound the viscosity of which is greater than 5 Pa·s⁻¹ as impregnating agent for the sensitive substance, and
a coating for the core, which contains at least one viscous compound the viscosity of which is greater than 5 Pa·s⁻¹ as sticking agent.

The sensitive substance cannot be an enzyme intended for non-human animal food.

By “stable powder composition” is meant compositions in which the sensitive substance undergoes less than 20% degradation when said powder composition is subjected to temperatures of 60° C. to 100° C. and/or pressures of 2×10⁶ to 10⁷ Pa and/or a relative humidity of 60% to 100%.

Advantageously, the viscous compound is chosen from vegetable gums or fermentation products, starchy bases, chitins or cellulose derivatives, such as carboxymethyl cellulose (CMC), methyl cellulose, ethyl cellulose, propyl cellulose, hydroxymethyl cellulose, hydroxyethyl cellulose, hydroxypropyl cellulose, hydroxypropyl methyl cellulose, ethyl hydroxyethyl cellulose, or microcrystalline cellulose.

Particularly advantageously, the viscous compound is carboxymethyl cellulose (CMC).

The sensitive substance can be chosen in particular from proteins, vitamins, bacteria, yeasts, antioxidants, carotenoids, essential oils or pharmacologically active substances such as antibiotics.

In an advantageous embodiment, the core contains at least one support of the sensitive substance.

This support can be chosen in particular from starch, flours, in particular wheat, corn, manioc or rice flour, talc, beet pulp, maltodextrin, salts such as calcium carbonate or corn distiller's grain.

In an advantageous embodiment, the impregnating agent and the sticking agent are identical.

Particularly advantageously, the impregnating agent and the sticking agent contain or are constituted by carboxymethyl cellulose (CMC).

In a particular embodiment, the powder composition is constituted by: 5 to 40%, preferably 10 to 30%, dry extract of a sensitive substance, 10 to 60%, preferably 20 to 50%, dry extract of a support, 5 to 40%, preferably 10 to 30%, dry extract of an impregnating agent and 5 to 30%, preferably 10 to 20%, dry extract of a sticking agent.

The present invention also relates to a method for preparing a stable powder composition containing particles comprising:
a core containing at least one substance sensitive to temperatures of 20° C. to 100° C. and/or pressures of 10⁶ to 10⁷ Pa and/or a relative humidity of 60% to 100% and at least one viscous compound the viscosity of which is greater than 5 Pa·s⁻¹ as impregnating agent for the sensitive substance, and
a coating for the core, which contains at least one viscous compound the viscosity of which is greater than 5 Pa·s⁻¹ as sticking agent
   said method comprising:
   a microgranulation stage for a mixture of said sensitive substance and said impregnating agent in order to obtain the core, and
   a stage of coating said core with said sticking agent.

The present invention also relates to a method for preparing a stable powder composition containing particles comprising:
a core containing at least one substance sensitive to temperatures of 20° C. to 100° C. and/or pressures of 10⁶ to 10⁷ Pa and/or a relative humidity of 60% to 100%, at least one viscous compound the viscosity of which is greater than 5 Pa·s⁻¹ as impregnating agent for the sensitive substance and at least one support of the sensitive substance, and
a coating for the core, which contains at least one viscous compound the viscosity of which is greater than 5 Pa·s⁻¹ as sticking agent,
   said method comprising:
   a microgranulation stage for a mixture of said sensitive substance, said support and said impregnating agent in order to obtain the core, and
   a stage of coating said core with said sticking agent.

During this method for preparing the stable powder composition, the core of the particles of this powder composition is obtained from a mixture of three ingredients, the sensitive substance, a support and the impregnating agent, by a microgranulation stage. The core of the particles is then coated with the sticking agent to form a protective film around the particle and thus protect the sensitive substance from the future stresses to which it will have to be subjected, in particular during the formation of granules in the granulating presses. This also makes it possible to preserve it during transport or storage.

The combination of the choice of the impregnating agent and the sticking agent, as well as of the technology implemented ensures that the stable powder composition obtained is comprised of particles stuck to a protective film providing it with novel properties. In particular, the powder composition has hydrophobic properties and the sensitive substance contained in said composition becomes temperature- and pressure-resistant and remains stable during transport or storage. However, the powder composition becomes rehydrated and therefore becomes bioavailable again when it is put back into solution.

The microgranulation stage can be carried out starting from a mixture of said sensitive substance and said support, which have previously been co-dried, and all of the impregnating agent.

During such a method, the previously co-dried sensitive substance and support are in the form of a completely homogeneous product, with a controlled particle size and dispersion, capable of mixing with the impregnating agent.

The microgranulation stage can also be carried out starting from a mixture of said sensitive substance, said support and all of the impregnating agent, which have previously been co-dried.

This method advantageously makes it possible to make direct use of a perfectly homogeneous product while reducing a costly mixing stage.

The microgranulation stage can also be carried out starting from a mixture of said sensitive substance, said support and some of the impregnating agent, which have previously been co-dried, and the remainder of the impregnating agent.

This method gives an intermediate powder, the particle sizes of which are controlled with a reduced dispersion. The majority of the impregnating agent is already present, so it is sufficient to top up with the remainder of the impregnating agent in order to start the following phase.

In a particular embodiment, said method comprises a co-drying stage, prior to the microgranulation stage, for a mixture containing the sensitive substance, the support of the sensitive substance and optionally all or some of the impregnating agent, the co-drying being carried out at an output air temperature of less than 60° C. and at a powder temperature of less than 45° C.

In a preferred embodiment of the method of the invention, the sensitive substance is used in liquid form and the support in dry form.

In another particular embodiment, said method comprises:
a co-drying stage for a mixture containing the sensitive substance and at least one support of the sensitive substance, the co-drying being carried out at an output air temperature of less than 60° C. and at a powder temperature of less than 45° C., in order to obtain an intermediate homogeneous powder,
a microgranulation stage for the intermediate homogeneous powder with the impregnating agent in order to obtain a densified microgranulated powder constituted by uncoated particles corresponding to the abovementioned cores,

In a preferred embodiment of the method of the invention, the sensitive substance is used in liquid form, the support in dry form and the impregnating agent in solid form.

The co-drying stage is carried out at a low temperature, which allows a retention of more than 95% of the sensitive substance and leads to a homogeneous powder being obtained, the average grain size of which measured as D(v,0.5) can be adjusted from 50 to 250 μm.

The intermediate homogeneous powder then contains 10 to 100%, in particular 40 to 65%, in particular 55%, sensitive substance in dry extract.

The final humidity of the intermediate homogeneous powder varies from 0% to in particular 5 to 12% and the water activity is less than 0.6 in order to avoid any development of microorganisms.

The microgranulation stage is generally carried out at a maximum temperature of 45° C. so as not to damage the sensitive substance and at a given water content, i.e. approximately 5 to 20% water added per total dry extract used and preferably 10%, so that the impregnation and the consistency of the product are achieved by controlling the homogeneity of the particles and by monitoring the level of humidity and by a visual check using a binocular magnifier or by image analysis.

The densified powder is characterized in that a change of state of the product is observed, in powder form at the start, due to the agglomeration of the particles, and this, just before obtaining the consistency of a pasty product, which is monitored either by the temperature which increases very rapidly or by the amperage consumption of the main motor of the microgranulator.

The densified powder obtained then has an average grain size measured as D(v,0.5) of 100 to 200 μm and a water content of 5 to 10%.

This densified powder is constituted by 5 to 50%, preferably 10 to 35%, sensitive substance in dry extract, by 10 to 70%, preferably 20 to 60%, support in dry extract and by 5 to 50%, preferably 10 to 30%, impregnating agent.

In a particular embodiment, the method of the invention for preparing the powder composition comprises:
a co-drying stage for a mixture containing the sensitive substance and at least one support of the sensitive substance, the co-drying being carried out at an output air temperature of less than 60° C. and at a powder temperature of less than 45° C., in order to obtain an intermediate homogeneous powder,
a microgranulation stage for the intermediate homogeneous powder with the impregnating agent in order to obtain a densified microgranulated powder constituted by uncoated particles corresponding to the abovementioned core,
a stage of coating the uncoated particles of the densified microgranulated powder with the sticking agent in order to obtain a coated powder.

In a preferred embodiment of the method of the invention, the sensitive substance is used in liquid form, the support in dry form, the impregnating agent in solid form and the sticking agent in liquid form, in particular in solution in water.

During the coating stage, the rate of deposition of sticking agent around the core of the particles depends on the desired final grain size of the stable powder composition. In particular it varies from 5 to 30%, preferably from 10 to 20%, dry extract of the powder composition.

The sticking agent is deposited in particular in liquid form, starting from a solution of 4 to 10% dry extract in water. The solution containing the sticking agent is such that it has a viscosity compatible with spraying allowing the densified powder to be coated grain by grain, without agglomeration of the particles.

The obtained coated powder has an average grain size measured as D(v,0.5) of 300 to 400 μm and a water content of 5 to 12%.

This coated powder is constituted by 5 to 40%, preferably 10 to 30%, sensitive substance in dry extract, by 10 to 60%, preferably 20 to 50%, support in dry extract, by 5 to 40%, preferably 10 to 30%, impregnating agent in dry extract and by 5 to 30%, preferably 10 to 20%, sticking agent in dry extract.

In another particular embodiment of the invention, the method comprises:
a co-drying stage for a mixture containing the sensitive substance and at least one support of the sensitive substance, the co-drying being carried out at an output air temperature of less than 60° C. and at a powder temperature of less than 45° C., in order to obtain an intermediate homogeneous powder,
a microgranulation stage for the intermediate homogeneous powder with the impregnating agent in order to obtain a densified microgranulated powder constituted by uncoated particles corresponding to the abovementioned core,
optionally a low-temperature drying stage for the densified microgranulated powder at a temperature of less than 45° C., in order to obtain a dried densified microgranulated powder,
optionally a sieving stage in order to obtain a sieved densified microgranulated powder,
a stage of coating the uncoated particles of the densified microgranulated powder, optionally dried and optionally sieved, with the sticking agent in order to obtain a coated powder
a drying stage for the coated powder in order to obtain a stable powder composition.

By “low-temperature drying” is meant a drying stage where the temperature does not exceed 45° C.

This drying stage is carried out after the microgranulation stage if the densified powder obtained after this microgranulation stage has a water content greater than 10% and in order to obtain a water content of less than 9%, and preferably less than 7%.

The sieving stage is carried out after the microgranulation stage in order to provide particles of equivalent size prior to coating.

The densified microgranulated powder, optionally dried and optionally sieved, obtained before the coating stage then has an average grain size measured as D(v,0.5) of 100 to 200 μm and a maximum water content of 10%.

The drying stage carried out after the coating stage is performed at a low temperature, i.e. at a maximum temperature of 45° C. in the core, in conditions appropriate to the sensitive substance and makes it possible to obtain a stable powder composition having a water content of less than 10%, and preferably less than 8%.

The grain size of the stable powder composition is mainly comprised within the range from 100 to 500 μm.

The stable powder composition thus obtained can be sold directly as raw material to be introduced in particular into granules for animal food purposes, since this particulate size is the one which avoids any demixing in the formulated products intended for animal food.

The viscous compound is advantageously chosen from vegetable gums or fermentation products, starchy bases, chitins or cellulose derivatives, such as carboxymethyl cellulose (CMC), methyl cellulose, ethyl cellulose, propyl cellulose, hydroxymethyl cellulose, hydroxyethyl cellulose, hydroxypropyl cellulose, hydroxypropyl methyl cellulose, ethyl hydroxyethyl cellulose, or microcrystalline cellulose.

Particularly advantageously, the viscous compound is carboxymethyl cellulose (CMC).

The sensitive substance can be chosen in particular from proteins, in particular enzymes, vitamins, bacteria, yeasts, antioxidants, carotenoids, essential oils or pharmacologically active substances such as antibiotics.

The support of the sensitive substance can be chosen in particular from starch, flours, in particular wheat, corn, manioc or rice flour, talc, beet pulp, maltodextrin, salts such as calcium carbonate, corn distiller's grain.

In an advantageous embodiment, the impregnating agent and the sticking agent are identical.

Particularly advantageously, the impregnating agent and the sticking agent contain or are constituted by carboxymethyl cellulose (CMC).

This method is more economically attractive on the one hand because CMC is inexpensive and on the other hand because this method makes it possible to obtain an increased resistance to temperature and pressure for a level of CMC (used as impregnating and sticking agent) which can be reduced to 35% or even down to 25% in dry extract of final powder composition (whereas conventionally it is 55%).

In a particular embodiment, the sensitive substance represents 5 to 40%, preferably 10 to 30%, dry extract of the powder composition, the support represents 10 to 60%, preferably 20 to 50%, dry extract of the powder composition, the impregnating agent represents 5 to 40%, preferably 10 to 30%, dry extract of the powder composition and the sticking agent represents 5 to 30%, preferably 10% to 20%, dry extract of the powder composition.

The present invention also relates to a powder composition obtained by a method of the invention as defined above.

The present invention also relates to a pharmaceutical composition comprising, as active substance, a stable powder composition containing particles comprising:
a core containing at least one substance sensitive to temperatures of 20° C. to 100° C. and/or pressures of 10⁶ to 10⁷ Pa and/or a relative humidity of 60% to 100% and at least one viscous compound the viscosity of which is greater than 5 Pa·s⁻¹ as impregnating agent for the sensitive substance, and
a coating for the core, which contains at least one viscous compound the viscosity of which is greater than 5 Pa·s⁻¹ as sticking agent,
   in which the sensitive substance has at least 80%, in particular at least 90%, of its initial activity,
   in combination with a pharmaceutically acceptable vehicle.

This pharmaceutical composition can be intended in particular for animals.

The present invention also relates to a food composition comprising a stable powder composition as defined in the present invention, in which the sensitive substance has at least 80%, in particular at least 90%, of its initial activity, and is not an enzyme intended for non-human animal food, and at least one food product.

This food composition can be intended in particular for animals.

This food composition can moreover be characterized in that it is presented in the form of pellets or granules.

The present invention also relates to a method for preparing food compositions comprising a stable powder composition as defined in the present invention, and at least one food product, said method comprising a moist compression stage, in particular at 14% humidity, with an applied pressure of 2×10⁶ to 10⁷ Pa, for a mixture of the stable powder composition with the food product.

In a particular embodiment, the abovementioned co-drying stage is performed continuously, in particular on a drying tower with atomization and/or fluidized bed.

The sensitive substance is introduced, during this stage, through nozzles arranged around the inlet for the dry support or by a central injection pipe, or a Shugi® or Bepex® type dynamic mixer (sold by HOSOKAWA) on industrial towers.

The co-drying stage can be performed in particular continuously by spraying the sensitive substance in the form of a monodisperse aerosol, directly on the support powder by an appropriate device consequently allowing a better effectiveness and perfectly homogeneous particles to be obtained.

In a particular embodiment, the abovementioned microgranulation stage takes place by moist microgranulation and shearing, in particular on a fluidized bed.

This stage consists in particular of impregnating the intermediate homogeneous powder with the impregnating agent and densifying it intermittently in a static mixer or continuously, for example in a SHUGI® dynamic mixer (sold by HOSOKAWA) using a rotating device such as a high-speed mixer for moist and pasty products (cutter) or a DIOSNA®, TURBOSPHERE® or GLATT® moist granulator used in pharmacy, (sold by DIOSNA, Pierre GUERIN and GLATT, respectively), fitted with a suitable spraying system.

In a preferred embodiment of the invention, the abovementioned micro granulation stage is performed continuously by wetting with a monodisperse aerosol, consequently allowing a better effectiveness and homogeneous particles to be obtained.

In a particular embodiment, the abovementioned low-temperature drying stage is performed either in a discontinuous fluidized bed, or directly if the impregnating stage takes place continuously, or on a drying tower using tube or Shugi co-drying devices.

In a particular embodiment, the abovementioned coating stage consists of depositing an adequate film of sticking agent, then drying the obtained product. The film is normally deposited by suitable spraying nozzles on fluidized air bed dryers.

In a preferred embodiment of the invention, the stage of coating by spraying a film is performed continuously with a monodisperse aerosol, consequently allowing a better effectiveness and homogeneous particles to be obtained in order to improve the density of the particles and limit the quantity of sticking agent deposited.

In a preferred embodiment of the invention, innovative systems for containing impregnating powder developed on INNOJET® equipment (sold by INNOJET) with ROTOJET® nozzles (sold by INNOJET) can be used to improve the density of the particles and limit the quantity of sticking agent deposited. This system makes it possible for the particles to pass homogeneously in front of the nozzle at a high flow rate.

The coated powder is then dried in successive batches on a standard GLATT or AEROMATIC bottom spray fluidized bed (sold by GLATT and GEA respectively), INNOJET®, top spray or tangential spray fluidized bed, or continuously with a continuous fluidized bed with a bank of nozzles or with a SHUGI agglomerator or in batches, the aim being to restore the water content of the coated powder to a value of less than 10% and preferably less than 8%.

### FIGURES

FIG. 1 schematically shows the different stages of the method for preparing a stable powder composition according to the invention, the raw materials being shown in a grey rectangle, the operations carried out being shown in a white diamond and the equipment and parameters used being shown in a grey oval. The code used is illustrated below

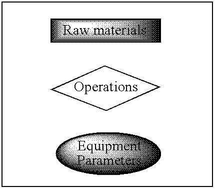

FIGS. 2 to 7 show different photographs of particles of a powder composition viewed through a scanning electron microscope.

FIGS. 2 (magnification ×50), 3 (magnification ×200) and 4 (magnification ×800) relate to particles obtained by a standard coating method on a fluidized bed.

FIGS. 5 (magnification ×50), 6 (magnification ×200) and 7 (magnification ×800) represent particles obtained by the method of the invention using innovative impregnating systems developed on INNOJET® equipment.

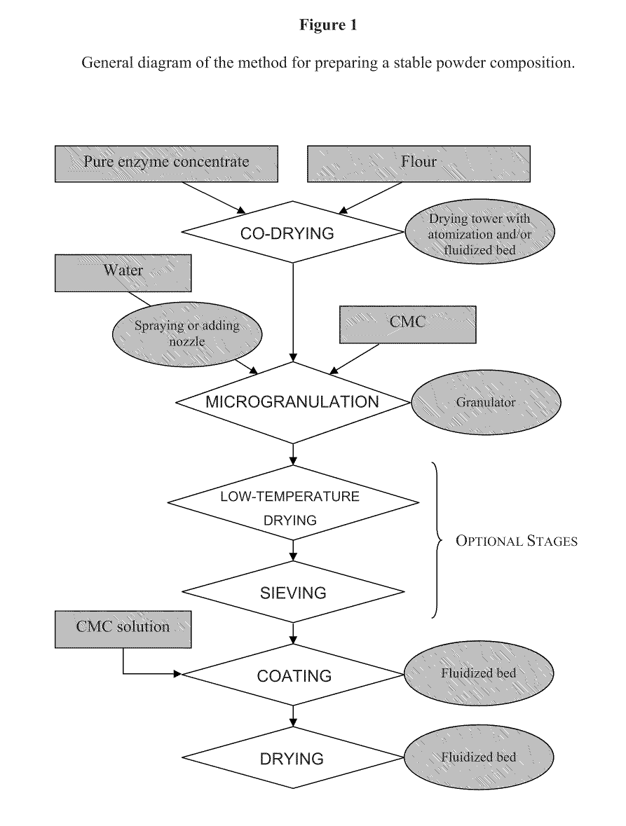

### EXAMPLES

### Example 1

### Preparation of a Stable Powder Composition Containing a Mixture of Enzymes

The experimental protocol below describes a method for preparing a stable powder composition in which the sensitive substance is a mixture of enzymes, the support is flour and the impregnating and sticking agents are constituted by CMC.

1. Co-Drying Stage

475 kg/h of enzyme solution with 28.7% dry matter (DM) is sprayed at 165.10⁵ Pa and co-dried continuously on a flour-type support, at a rate of 113 kg/h, at a low temperature:
input air temperature: 146° C.
output air temperature: 49° C.
temperature of the air in the static bed: 40° C.
temperature of the air in the vibro-fluidizer (first section): 35° C.
temperature of the air in the vibro-fluidizer (second section): 27° C.

The distribution of the dry extract is therefore as follows: 55% mixture of enzymes and 45% wheat flour used as support.

The mixture of enzymes is a concentrated filtered fermentation wort obtained from the fermentation of Penicillium funiculosum (IMI 378536) which contains 19 enzymatic activities, the most important of which are cellulase, xylanase and β-glucanase.

The Penicillium funiculosum used is protected by the patent EP 1 007 743 and was deposited on 24 Mar. 1998 by ADISSEO under number IMI 378536 at the IMI (International Mycological Institute, Bakeham Lane, Englefield Green, Egham, Surrey, TW20 9TY, UK), an international registration authority recognized under the Treaty of Budapest.

The homogeneous powder obtained in this stage has a powder character without fines smaller than 63 μm, which makes it a product capable of being treated in a second phase of impregnating said powder with an impregnating agent. The average grain size as D(v,0.5) is 117 μm and the humidity is 7.5%.

Using these devices, a strong bond is produced between the support and the liquid containing the enzyme, even in the case of products difficult to granulate such as wheat or corn flour.

This stage makes it possible to obtain a pre-stabilized, dry, intermediate, homogeneous powder.

2. Stage of Impregnation with the Impregnating Agent

The impregnation is carried out in a high-speed mixer for moist and pasty products (cutter) and comprises the following stages:
30 seconds pre-mixing the ingredients (0.77 kg of dry CMC extract per kilogram of dry intermediate homogeneous powder extract),
120 seconds adding water, at a rate of 0.18 kg of water per kilogram of dry intermediate powder extract, accompanied by stirring, and
120 seconds final mixing.

The object of this stage is to obtain particles of the order of 150 to 200 μm on average, with a humidity of 14%, while keeping the product at a temperature of less than 45° C. in order not to degrade the sensitive substance.

The product is thus dried on a fluidized bed dryer with an input temperature of 55° C. and an output temperature of 25° C.

The densified microgranulated powder obtained has a humidity of 7 to 8%.

3. Stage of Coating with the Sticking Agent

An aqueous solution of 7 to 7.5% CMC is heated to a temperature of 60° C. to 70° C., before being sprayed onto the densified microgranulated powder. The quantity of CMC deposited is 0.25 kg of dry CMC extract per kilogram of dry densified microgranulated powder extract to be coated.

The powder obtained in this stage has an average grain size of 300 to 400 μm and a humidity of 10 to 11%.

The coated product is then dried continuously on a fluidized bed or in batches, the aim being to bring the product to a water content of less than 10% and preferably less than 8%.

The stable powder composition obtained as end product then has the following composition: 25% dry extract of enzymes, 20% dry support extract, 55% dry CMC extract (35% being used for the impregnating stage and 20% being used for the coating stage).

Moreover, the powder composition has a water content of 7.6%, a density of 450 g/l and the following grain size distribution:
greater than 800 μm→0%
between 500 and 800 μm→12%
between 300 and 500 μm→46%
between 200 and 300 μm→26%
between 100 and 200 μm→16%
less than 100 μm→0%

4. Results

The properties of the powder obtained at the end of each stage are summarized in Table 1 below.

TABLE 1

| Stage | Diameter (in μm) | Level of humidity |
|---|---|---|
| Co-drying | 117 on average | 7.5% |
| (intermediate powder) | | |
| Impregnation | 150-200 | before drying: 14% |
| (densified powder) | | after drying: 7 to 8% |
| Coating | 300-400 | before drying: 10 to 11% |
| (stable powder | | after drying: <10%, |
| composition) | | in particular <8% |

### Example 2

### Preparation of a Stable Powder Composition Containing a Superoxy Dismutase (SOD) Enzyme

The experimental protocol below describes a method for preparing a stable powder composition in which the sensitive substance is a superoxy dismutase enzyme, the support is wheat maltodextrin and the impregnating and sticking agents are constituted by CMC.

1. Co-Drying Stage

95 kg/h of enzymatic solution with 9.5% dry matter (DM) is sprayed at 95.10⁵ Pa and co-dried continuously on a maltodextrin type support, at a rate of 9.5 kg/h, at a low temperature:
input air temperature: 110° C.
output air temperature: 50° C.
temperature of the air in the static bed: 40° C.

The distribution of the dry extract is therefore as follows: 50% pure enzyme and 50% wheat maltodextrin support.

The average grain size of the obtained powder as D(v,0.5) is 95 μm and the humidity is 4.5%.

This stage makes it possible to obtain a pre-stabilized, thy, intermediate, homogeneous powder.

2. Stage of Impregnation with the Impregnating Agent

The impregnation is carried out in a high-speed mixer for moist and pasty products (cutter) and comprises the following stages:
60 seconds pre-mixing the ingredients (0.25 kg of dry CMC extract per kilogram of dry intermediate powder extract),
90 seconds adding water, at a rate of 1 kg of water per kilogram of dry intermediate powder extract, accompanied by stirring, and
90 seconds final mixing.

The product is dried on a fluidized bed dryer with an input temperature of 60° C. and an output temperature of 40° C.

The densified microgranulated powder obtained has a humidity of 8%.

3. Stage of Coating with the Sticking Agent

An aqueous solution of 7% CMC is heated to a temperature of 60° C. to 70° C. The quantity of CMC deposited is 0.5 kg of dry CMC extract per kilogram of dry densified microgranulated powder extract to be coated.

The powder obtained in this stage has an average grain size of 300 μm and humidity level of 9%.

The stable powder composition obtained as end product then has the following composition: 20% dry extract of enzyme, 20% dry support extract, 60% dry CMC extract (10% being used for the impregnating stage and 50% being used for the coating stage).

Moreover, the powder composition has a water content of 9%, a density of 400 g/l and the following grain size distribution:
greater than 800 μm→0%
between 500 and 800 μm→7%
between 300 and 500 μm→35%
between 200 and 300 μm→38%
between 100 and 200 μm→20%
less than 100 μm→0%

### Example 3

### Preparation of a Stable Powder Composition Containing a Bacterium

The experimental protocol below describes a method for preparing a stable powder composition in which the sensitive substance is a bacterium of the lactobacillus type, the support and the impregnating agent are Perfectamyl® starch and the sticking agent is constituted by CMC.

1. Co-Drying Stage

105 kg/h of a fermentation medium with 10% dry matter (DM) containing 10⁹ bacteria per gram is sprayed at 50.10⁵ Pa and co-dried continuously on a Perfectamyl® starch support, at a rate of 45 kg/h, at a low temperature:
input air temperature: 90° C.
output air temperature: 50° C.

The distribution of the dry extract is therefore as follows: 20% dry extract of pure bacterium and 80% starch support.

The average grain size of the obtained powder as D(v,0.5) is 45 μm and the humidity is 4%.

This stage makes it possible to obtain a pre-stabilized, dry, intermediate, homogeneous powder.

2. Stage of Impregnation with the Impregnating Agent

The impregnation is carried out in a high-speed mixer in a controlled and climatized atmosphere and comprises the following stages:
30 seconds pre-mixing the ingredients (0.2 kg of dry CMC extract per kilogram of dry intermediate powder extract)
30 seconds adding water, at a rate of 0.4 kg of water per kilogram of dry intermediate powder extract, accompanied by stirring, and
30 seconds final mixing.

The product is dried on a fluidized bed dryer with input air with less than 2 g of water per kilogram of air, an input temperature of 50° C. and a maximum output temperature of 40° C.

The densified microgranulated powder obtained has a humidity of 7%.

3. Stage of Coating with the Sticking Agent

An aqueous solution of 7% CMC is heated to a temperature of 60° C. to 70° C. The quantity of CMC deposited is 0.66 kg of dry CMC extract per kilogram of dry densified microgranulated powder extract to be coated.

The powder obtained in this stage has an average grain size of 350 μm and a humidity of 7% and a water activity of less than 0.2 in order to stabilize the bacteria in the core.

The stable powder composition obtained as end product then has the following composition: 10% dry extract of the pure bacteria fermentation medium, 50% dry extract of starch (40% being used as support and 10% as impregnating agent) and finally 40% dry CMC extract used as sticking agent.

Moreover, the powder composition has a water content of 7% and a density of 450 g/l.

### Example 4

### Preparation of a Stable Powder Composition Containing an Antibiotic

The experimental protocol below describes a method for preparing a stable powder composition in which the sensitive substance is an antibiotic, which is tylosin, already in powder form, the support is flour and the impregnating agent and the sticking agent are constituted by CMC.

1. Co-Mixing Stage

10 kg of a tylosin antibiotic in powder form with 96.4% (DM) is mixed with 13 kg of wheat flour.

The distribution of the dry extract is therefore as follows: 44% dry extract of pure antibiotic and 56% flour.

The average grain size of the obtained powder as D(v,0.5) is 40 μm and the humidity is 4.7%.

This stage makes it possible to obtain a dry, intermediate, homogeneous powder pre-stabilized vis-à-vis water activity.

2. Stage of Impregnation with the Impregnating Agent

The impregnation is carried out in a high-speed mixer in a controlled and climatized atmosphere and comprises the following stages:
90 seconds pre-mixing the ingredients (0.4 kg of dry CMC extract per kilogram of dry intermediate powder extract)
60 seconds adding water, at a rate of 0.1 kg of water per kilogram of dry intermediate powder extract, accompanied by stirring, and
60 seconds final mixing.

The product is dried on a fluidized bed dryer with input air with less than 2 g of water per kilogram of air, an input temperature of 60° C. and a maximum output temperature of 50° C.

The densified microgranulated powder obtained has a humidity of 7%.

3. Stage of Coating with the Sticking Agent

An aqueous solution of 7% CMC is heated to a temperature of 60° C. to 70° C. The quantity of CMC deposited is 0.14 kg of dry CMC extract per kilogram of dry densified microgranulated powder extract to be coated.

The powder obtained in this stage has an average grain size of 500 μm and a humidity of 5.6% and a water activity of less than 0.2 in order to stabilize the antibiotic in the mixtures during application.

The stable powder composition obtained as end product then has the following composition: 27.5% dry extract of antibiotic, 35% dry support extract, 37.5% dry CMC extract (25% being used for the impregnating stage and 12.5% being used for the coating stage).

Moreover, the stable powder composition has a density of 450 g/l.

### Example 5

### Preparation of a Stable Powder Composition Containing a Mixture of Carotenoids

The experimental protocol below describes a method for preparing a stable powder composition in which the sensitive substance is a mixture of carotenoids containing in particular 55% astaxanthin, the support is calcium carbonate and the impregnating and sticking agents are constituted by CMC.

1. Co-drying stage

400 kg/h of an astaxanthin-rich fermentation medium with 15% dry matter (DM) is sprayed at 100.10⁵ Pa and co-dried continuously on a support of the calcium carbonate type, at a rate of 20 kg/h, at a low temperature:
input air temperature: 130° C.
output air temperature: 50° C.
temperature of the air in the static bed: 40° C.

The distribution of the dry extract is therefore as follows: 75% dry matter of the carotenoids solution and 25% calcium carbonate support.

The average grain size of the obtained powder as D(v,0.5) is 105 μm and the humidity is 5%.

This stage makes it possible to obtain a pre-stabilized, dry, intermediate, homogeneous powder.

2. Stage of Impregnation with the Impregnating Agent

The impregnation is carried out in a high-speed mixer for moist and pasty products (cutter) and comprises the following stages:
90 seconds pre-mixing the ingredients (0.5 kg of dry CMC extract per kilogram of dry intermediate powder extract),
90 seconds adding water, at a rate of 1 kg of water per kilogram of dry intermediate powder extract, accompanied by stirring, and
180 seconds final mixing.

The product is dried on a fluidized bed dryer with an input temperature of 70° C. and an output temperature of 50° C.

The densified microgranulated powder obtained has a humidity of 8%.

3. Stage of Coating with the Sticking Agent

An aqueous solution of 7% CMC is heated to a temperature of 60° C. to 70° C. The quantity of CMC deposited is 0.25 kg of dry CMC extract per kilogram of dry densified microgranulated powder extract to be coated.

The powder obtained in this stage has an average grain size of 300 μm and a humidity of 8%.

The stable powder composition obtained as end product then has the following composition: 40% dry extract of the carotenoids solution, 13% dry extract of calcium carbonate as support, 47% dry CMC extract (27% being used for the impregnating stage and 20% being used for the coating stage).

Moreover, the stable powder composition has a water content of 8% and a density of 510 g/l.

### Example 6

### Preparation of a Stable Powder Composition Containing an Essential Oil

The experimental protocol below describes a method for preparing a stable powder composition in which the sensitive substance is an essential oil, the support is wheat flour, the impregnating agent and the sticking agent are constituted by CMC.

1. Co-Mixing Stage

50 kg/h of an essential oil of garlic is sprayed at 150.10⁵ Pa and co-mixed continuously on a wheat flour type support, at a rate of 54 kg/h, at a low temperature:
input air temperature: 20° C.

The distribution of the dry extract is therefore as follows: 50% dry matter of the essential oil of garlic and 50% wheat flour support.

The average grain size of the obtained powder as D(v,0.5) is 50 μm and the humidity is 7%.

This stage makes it possible to obtain a pre-stabilized, intermediate, homogeneous powder.

2. Stage of Impregnation with the Impregnating Agent

The impregnation is carried out in a high-speed mixer for moist and pasty products (cutter) and comprises the following stages:
120 seconds pre-mixing the ingredients (0.15 kg of dry CMC extract per kilogram of dry intermediate powder extract),
90 seconds adding water, at a rate of 1 kg of water per kilogram of dry intermediate powder extract, accompanied by stirring, and
180 seconds final mixing.

The product is dried on a fluidized bed dryer with an input temperature of 50° C. and an output temperature of 40° C.

The densified microgranulated powder obtained has a humidity of 7%.

3. Stage of Coating with the Sticking Agent

An aqueous solution of 7% CMC is heated to a temperature of 60° C. to 70° C. The quantity of CMC deposited is 0.18 kg of dry CMC extract per kilogram of dry densified microgranulated powder extract to be coated.

The powder obtained in this stage has an average grain size of 450 μm and a humidity of 8%.

The stable powder composition obtained as end product then has the following composition: 37.5% dry extract of essential oil of garlic, 37.5% dry extract of wheat flour as support, 25% dry CMC extract (10% being used for the impregnating stage and 15% being used for the coating stage).

Moreover, the stable powder composition has a water content of 8% and a density of 550 g/l.

### Example 7

### Microscopic Characterization of the Stable Powder Composition Containing a Mixture of Enzymes

1. Experimental Protocol

Two types of stable powder composition were observed using the scanning electron microscope:
a powder composition obtained using a standard coating method of the GLATT® or AEROMATIC® type, containing 55% CMC as a percentage of the dry extract, and
a powder composition obtained using a coating method on innovative impregnating systems developed on INNOJET® equipment, containing 35% CMC as a percentage of the dry extract, with the ROTOJET® nozzles.

The standard coating method consists of covering a solid support using a layer of product by suspending the material to be coated in an air current and by spraying the coating liquid into the built-up fluidized bed.

The coating method using innovative systems consists, in turn, of spraying the sticking agent, in order to form a protective film, continuously with a monodisperse aerosol, consequently allowing a better effectiveness and homogeneous particles to be obtained in order to improve the density of the particles and limit the quantity of sticking agent deposited. This system makes it possible for the particles to pass homogeneously in front of the nozzle at a high flow rate.

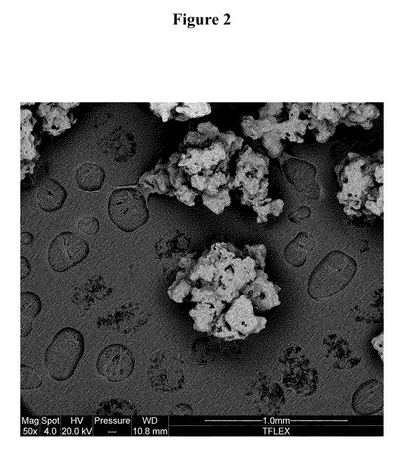

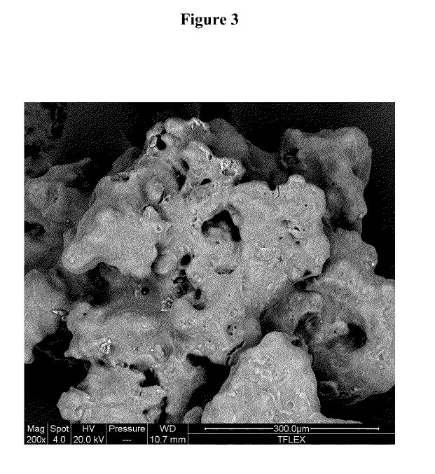

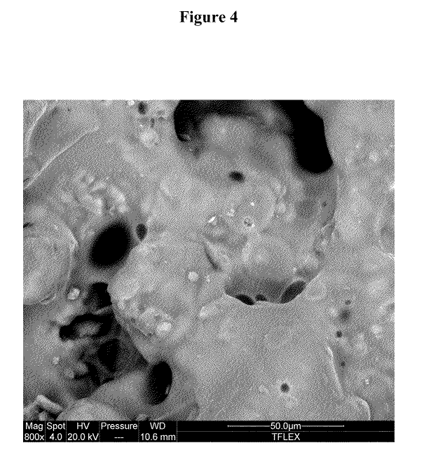

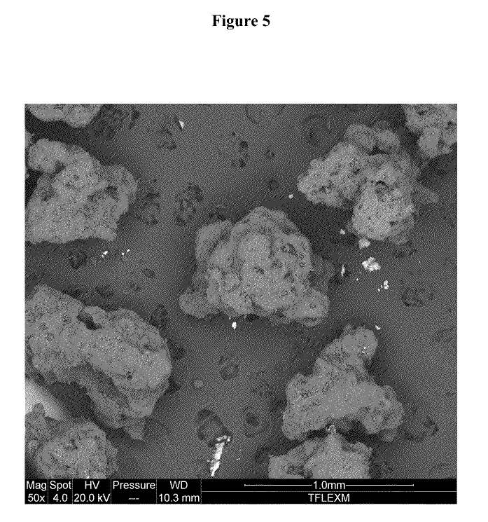

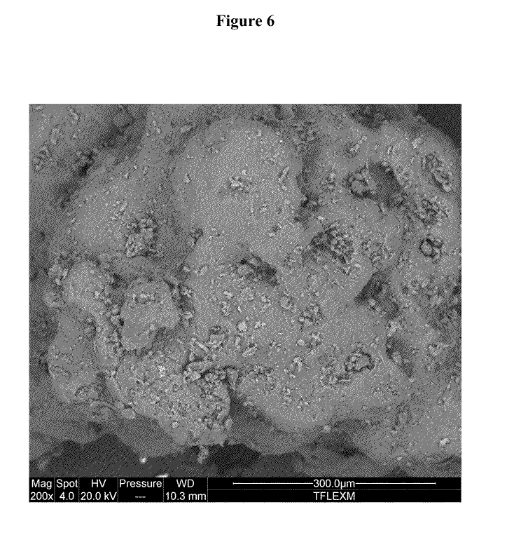

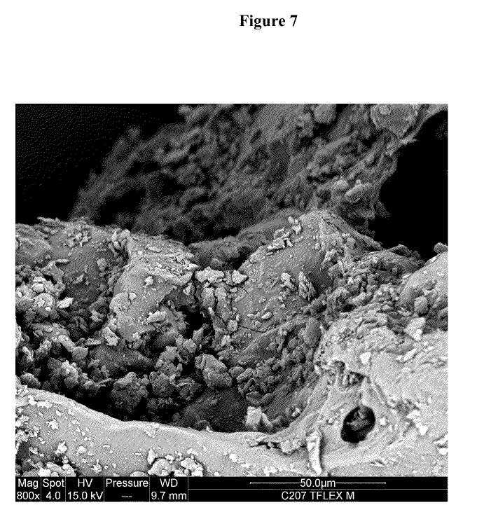

It is thus observed that the deposition of the sticking agent is more regular, compacted and densified with the innovative method (see FIGS. 5 to 7) than with a standard coating method (see FIGS. 2 to 4), consequently making it possible to reduce the quantity of CMC used, in an advantageous manner.

2. Results

The protective film (or coating) covering the core of the particles is visible with the scanning electron microscope.

In the case of a standard coating method, a heterogeneity of the particles is noted, with the presence of partial voids, whence it is difficult to deposit the sticking agent in the voids created (see FIGS. 2, 3 and 4).

The particles obtained by a coating method on innovative impregnating systems, which therefore contain less CMC, are more compacted and dense (see FIGS. 5, 6 and 7).

### Example 8

### Resistance to Temperature and Pressure of the Enzymes Contained In the Stable Powder Composition

1. Experimental Protocol for Preparing Granules

The objective is to test the enzymatic activity of preparations constituted by powder compositions containing enzymes added to a base of growth-promoting meal-based poultry feed after a granulation stage.

Three conditioner output processing temperatures (80, 85 and 90° C.) are used for each mixture according to a protocol described below.

1.1. Preparations Containing the Enzymes

Test 1: A 60-kg mixture produced with 59.998 kg of growth-promoting meal-based poultry feed and 3 g of a powder composition is prepared using a mixer with horizontal blades rotating at 60 rpm. The mixing lasts 2 minutes.

Test 2: A 500-g pre-mixture is produced with 2 g of a powder composition and 498 g of growth-promoting meal-based poultry feed.

A 40-kg mixture produced with 39.500 kg of growth-promoting meal-based poultry feed and the above pre-mixture is prepared using a mixer with horizontal blades rotating at 60 rpm. The mixing lasts 2 minutes.

The powder composition used then consists of:
a standard composition without protection for the products A and C corresponding to the product ROVABIO EXCEL AP sold by ADISSEO France SAS, which contains 80% wheat flour and 20% sensitive substance, and
a composition according to the invention with protection containing 20% sensitive substance, 25% wheat flour support and 55% CMC (used as impregnating agent and sticking agent) for the product B or 35% CMC for the products D and E,
   the rate of incorporation of said powder composition into the growth-promoting meal-based poultry feed being 50 g/T.

These different compositions were prepared from the same batch of sensitive substance, namely a concentrated filtered fermentation wort obtained from Penicillium funiculosum (IMI 378536) which contains 19 enzymatic activities, the most important of which are xylanase, β-glucanase and cellulase, and on which the commercial product ROVABIO EXCEL AP is based.

The above mixture (Test 1 or 2) is emptied into a rectangular container before being placed in bags. A representative sample of the mixture of approximately 1 kg is taken, in groups of 20 samples obtained by quartering in the rectangular container.

1.2. Granulation Stages

The granulation tests are performed on a laboratory press with a flat die (3 kW KAHL 14-175 press). The press die used has channels 4 mm in diameter and 24 mm thick (compression rate: 6).

For each granulation test, a laboratory cooler-dryer is used to dry and cool the samples of hot granules collected leaving the die. The cooling-drying time is at least 5 minutes for a load of hot granules of approximately 3.5 kg per cooler.

A representative sample of each manufacture of granules of approximately 500 g is taken.

1.3. Measurements and Taking of Samples Intended for Checking the Granulation

The measurements and samples are taken during the stabilized operation of the press (constant flow rate, stable electric power consumption and temperatures).

The measurements of humidity and residence time in the die are taken based on samples taken during the stabilized phase of each test.

The characterizations and press operating parameters are noted and recorded during the tests (see the attached granulation charts).

Steam Measurement:

The steam pressure and flow rate are recorded once a second by acquisition software.

The opening of the control valve for the vapour is noted manually on the control unit.

Measurement of the Temperature and Humidity:

The temperatures (ambient air, preparation before and after conditioner, die) are recorded once a second by acquisition software.

The temperature of the die corresponds to the temperature of the granules.

The humidity measurements are taken after drying a 5-g sample in the oven at 103° C. for 4 hours. All of the measurements are taken twice.

Measurement of the Set Point of the Bis Screw Feeder:

This measurement is taken manually in the course of each test.

Measurement of the Press Output:

The press output is measured by weighing a sample leaving the die over 30 seconds.

Measurement of the Electric Power Consumed:

This measurement, calculated by a power converter, is recorded once a second by acquisition software.

With the output, this parameter makes it possible to calculate the net specific production (kg/kWh) and the net specific consumption (kWh/T).

Measurement of the Residence Times of the Granules in the Die:

This measurement is calculated in relation to the output of the press.

The calculation takes account of the weight of 20 cm of granules.

The granulation parameters are given in the following summary tables and summarized for each conversion phase of the product.

TABLE 2
Preparation on entering the conditioner

| | Processing | Bis screw | Temperature | Humidity |
|---|---|---|---|---|
| Test | temperature | feeder (mV)* | (° C.)** | (%)*** |
| A | 80° C. | 37 | 18.6 | 10.7 |
| B | | 37 | 18.8 | 10.9 |
| C | | 37 | 17.8 | 10.1 |
| D | | 37 | 17.9 | 10.0 |
| E | | 37 | 17.9 | 10.2 |
| A | 85° C. | 38 | 19.2 | 10.7 |
| B | | 37 | 19.2 | 10.9 |
| C | | 36 | 18.3 | 10.1 |
| D | | 36 | 18.4 | 10.0 |
| E | | 36 | 18.3 | 10.2 |
| A | 90° C. | 37 | 19.5 | 10.7 |
| B | | 37 | 19.6 | 10.9 |
| C | | 36 | 19.0 | 10.1 |
| D | | 36 | 18.9 | 10.0 |
| E | | 36 | 18.9 | 10.2 |

*value read during each test
**average over the course of recording the test (1 value every second)
***average of two measurements

TABLE 3
Preparation on leaving the conditioner

| | | | Vapour | | |
|---|---|---|---|---|---|
| | Processing | Temperature | pressure | Vapour flow | Humidity |
| Test | temperature | (° C.)* | (10⁵ Pa)* | rate (kg/h)* | (%)** |
| A | 80° C. | 80.4 | 1.6 | 4.5 | 15.4 |
| B | | 80.2 | 1.6 | 4.5 | 15.2 |
| C | | 80.1 | 1.6 | 4.7 | 14.8 |
| D | | 80.1 | 1.6 | 4.7 | 14.7 |
| E | | 80.2 | 1.6 | 4.5 | 14.7 |
| A | 85° C. | 85.1 | 1.6 | 4.8 | 15.7 |
| B | | 85.4 | 1.6 | 4.8 | 15.5 |
| C | | 85.1 | 1.6 | 5.2 | 15.0 |
| D | | 85.3 | 1.6 | 5.0 | 14.8 |
| E | | 85.0 | 1.6 | 5.2 | 14.9 |
| A | 90° C. | 90.1 | 1.6 | 5.6 | 16.3 |
| B | | 90.4 | 1.6 | 5.6 | 15.8 |
| C | | 90.0 | 1.6 | 5.8 | 15.3 |
| D | | 90.2 | 1.6 | 5.5 | 15.2 |
| E | | 90.0 | 1.6 | 5.5 | 15.4 |

*average over the course of recording the test (1 value every second)
**average of two measurements

TABLE 4
Granules leaving the press

| | | | Die input/ | Die | |
|---|---|---|---|---|---|
| | | Die | output | residence | Press |
| | Processing | temperature | difference | time | output |
| Test | temperature | (° C.)* | (° C.)** | (s)*** | (kg/h)**** |
| A | 80° C. | 80.7 | 0.3 | 7.3 | 42.3 |
| B | | 80.7 | 0.5 | 7.6 | 40.6 |
| C | | 80.1 | 0.0 | 7.3 | 41.7 |
| D | | 80.8 | 0.7 | 7.4 | 41.5 |
| E | | 80.0 | 0.8 | 7.4 | 41.7 |
| A | 85° C. | 83.4 | −1.7 | 7.4 | 40.7 |
| B | | 83.4 | −2.0 | 7.3 | 42.0 |
| C | | 84.5 | −0.6 | 7.5 | 40.3 |
| D | | 84.5 | −0.8 | 7.4 | 41.0 |
| E | | 84.6 | −0.4 | 7.5 | 40.8 |
| A | 90° C. | 85.7 | −4.4 | 7.4 | 40.9 |
| B | | 85.9 | −4.5 | 7.4 | 41.4 |
| C | | 86.6 | −3.4 | 7.2 | 41.8 |
| D | | 86.8 | −3.4 | 7.4 | 40.9 |
| E | | 86.8 | −3.2 | 7.4 | 41.0 |

*average over the course of recording the test (1 value every second)
**calculated over the course of recording the test (temperature of the die − temperature of the preparation on leaving the conditioner)
***calculated from the weight of 20 cm of granules
****measured over a sampling of 30 seconds

The regulating set points of the press are as follows:

Flow rate: approximately 41 kg/h,
Processing temperatures on output from conditioner: 80, 85 and 90° C.,
Vapour pressure: 1.6×10⁵ Pa,
Setting of the cutting height of the knife: 10 mm.

2. Experimental Protocol for Measuring the Enzymatic Activity

2.1. β-glucanase by the DNS Method

The test is based on the enzymatic hydrolysis of β-glucan from barley, a β-1,3(4)-glucan. The reaction products are determined by colorimetry by measuring the increase in the reducing groups using 3,5-dinitrosalicylic acid (DNS). The reducing-sugar concentration available after enzymatic hydrolysis is determined using a standard glucose curve, the absorbance of which is measured at 540 nm. The enzymatic activity calculated is then expressed in glucose equivalents.

A solution containing 1 ml of a 1% β-glucan solution (m/V) in a 0.1 M sodium acetate buffer at pH 5.0 and 1 ml solution of the enzyme at the appropriate level of dilution is incubated at 50° C. for 10 minutes. The enzymatic reaction is stopped by adding 2 ml of a DNS solution (1% (m/V) 3,5-dinitrosalicylic acid, 1.6% (m/V) NaOH, 30% (m/V) potassium and sodium tartrate (+) in distilled water). The solution is homogenized then placed in a water bath boiling at a minimum of 95° C. and then cooled down in a bath to room temperature (over 5 minutes). Ten millilitres of ultrapure water is added to the solution and the absorbance is measured at 540 nm in a glass cell having an optical path length of 1 cm.

The absorbance is corrected with that obtained for a reference solution to which the DNS is added before the enzyme solution.

The results are converted to μmoles of reducing sugar by comparison with a variety of standard solutions ranging from 0.00 to 0.04% (m/V) glucose, processed with DNS as in the case of the tests.

A unit of endo-1,3(4)-β-glucanase activity is defined as the quantity of enzyme which produces 1 μmole of glucose equivalent per minute and per gram of product, under the test conditions (pH 5.0 and 50° C.).

2.2. Xylanase by the DNS Method

The test is based on the enzymatic hydrolysis of birch xylan, a xylose polymer containing β-D-1,4 linkages. The reaction products are determined by colorimetry by measuring the increase in the reducing groups using 3,5-dinitrosalicylic acid. The reducing-sugar concentration available after enzymatic hydrolysis is determined using a standard xylose curve the absorbance of which is measured at 540 nm. The enzymatic activity calculated is then expressed in xylose equivalents.

A solution containing 1 ml of a 1% birch xylan solution (m/V) in a 0.1 M sodium acetate buffer at pH 5.0 and 1 ml solution of the enzyme at the appropriate dilution is incubated at 50° C. for 10 minutes. The enzymatic reaction is stopped by adding 2 ml of a DNS solution (1% (m/V) 3,5-dinitrosalicylic acid, 1.6% (m/V) NaOH, 30% (m/V) potassium and sodium tartrate (+) in distilled water). The solution is homogenized then placed in a water bath boiling at a minimum of 95° C. and then cooled down in a bath to room temperature (over 5 minutes). Ten millilitres of ultrapure water is added to the solution and the absorbance is measured at 540 nm in a glass cell having an optical path length of 1 cm.

The absorbance is corrected with that obtained for a reference solution to which the DNS is added before the enzyme solution.

The results are converted to μmoles of reducing sugar by comparison with a variety of standard solutions ranging from 0.00 to 0.04% (m/V) xylose, processed with DNS as in the case of the tests.

A unit of endo-1,4-β-xylanase activity is defined as the quantity of enzyme which produces 1 μmole of xylose equivalent per minute and per gram of product, under the test conditions (pH 5.0 and 50° C.).

2.3. Xylanase by the Viscosimetry Method

This method is specific to the determination of the endo-1,4-β-xylanase activity in the foods. The endo-1,4-β-xylanase hydrolyzes the xylosidic linkages of the xylan. The test is based on the enzymatic hydrolysis of the xylosic linkages of a solution of arabinose from wheat, β-1,4-xylan polysaccharide substituted with arabinose. The enzymatic activity is proportional to the reduction in viscosity of a solution of wheat arabinoxylan in the presence of the enzyme to be assayed.

A unit of endo-1,4-β-xylanase activity is defined as the quantity of enzyme which will hydrolyze the substrate, reducing the viscosity of the solution, in order to produce a change in the relative fluidity of 1 dimensionless unit per minute under the analysis conditions: pH 5.5 and 30° C.

3. Results

TABLE 5
Checking the enzymatic activities of the powder compositions (before
passing through the press)

| | Bruxel SD | Standard | Powder compositions | | |
|---|---|---|---|---|---|
| Present in the | concentrate | product | of the invention | | |
| preparation | — | A and C | B | D | E |
| Enzymatic | | | | | |
| activity (units/g) | | | | | |
| DNS xylanase | 6507 | 3810 | 4027 | 5344 | 5304 |
| Visco. xylanase | 39065 | 31053 | 30293 | 32273 | 31153 |
| DNS β-glucanase | 7626 | 4676 | 5265 | 6300 | 6081 |

These results show that the main enzymatic activities are preserved in the different powder compositions, with reference to the initial enzyme concentrate (Bruxel SD concentrate).

TABLE 6
Rate of enzymatic activity* recovery measured on the foods after
passing through the press at different temperatures

| % retention of the | Processing | | | Level of |
|---|---|---|---|---|
| enzymatic activity | temperature | | | CMC |
| in the end product after | of the press | | | as % of the |
| passing through the press | 80° C. | 85° C. | 90° C. | dry extract |
| Test 1 | | | | |
| Control product without | 76% | 68% | 54% | 0% |
| protection (A) | | | | |
| Product with protection (B) | 100% | 85% | 82% | 55% |
| Test 2 | | | | |
| Control product without | 86% | 57% | 40% | 0% |
| protection (C) | | | | |
| Product with protection (D) | 99% | 90% | 81% | 35% |
| Product with protection (E) | 96% | 86% | 83% | 35% |

*xylanase activity measured by viscometry

The results listed in Table 6 above demonstrate the stability that the powder composition according to the invention gives to the sensitive substance.

At 80° C., the enzyme retains 100% of its activity, whereas the enzyme that is not protected has lost ¼ of its activity (Test 1). At temperatures greater than 85° C., the enzyme retains more than 80% of its activity.

It is noted that an equivalent protection is obtained with Tests 1 and 2, which correspond to powder compositions containing 55% and 35% CMC respectively. The greater compaction and densification of the particles obtained with 35% CMC thus make it possible to retain the stability of the enzyme even with a smaller quantity of CMC.

It is also possible to envisage reducing the percentage of CMC used, and in particular decreasing it to 20% CMC, while keeping the same level of protection for the enzymes.

1-25. (canceled)

26. A method of substantially preventing degradation of a substance sensitive to temperatures of 20° C. to 100° C. and/or pressures of 10⁶ to 10⁷ Pa and/or a relative humidity of 60% to 100%, contained in a powder composition containing particles, said method comprising
including at least one viscous compound, the viscosity of which is greater than 5 Pa·s-1, wherein said powder composition contains particles comprising:
a core containing said sensitive substance and at least one viscous compound as impregnating agent for the sensitive substance, an
a coating for the core, which contains at least one viscous compound as a sticking agent.

27. The method according to claim 26, wherein the viscous compound is selected from the group consisting of vegetable gums, fermentation products, starchy bases, chitins and cellulose derivatives selected from the group consisting of carboxymethyl cellulose (CMC), methyl cellulose, ethyl cellulose, propyl cellulose, hydroxymethyl cellulose, hydroxyethyl cellulose, hydroxypropyl cellulose, hydroxypropyl methyl cellulose, ethyl hydroxyethyl cellulose, or microcrystalline cellulose.

28. The method according to claim 26, wherein the core contains at least one support of the sensitive substance.

29. The method according to claim 28, wherein the sensitive substance represents 5 to 40% dry extract of the powder composition, the support represents 10 to 60% dry extract of the powder composition, the impregnating agent represents 5 to 40% dry extract of the powder composition and the sticking agent represents 5 to 30% dry extract of the powder composition.

30. The method according to claim 28, wherein the sensitive substance is constituted by a mixture of enzymes constituted principally by β-glucanase, xylanase and cellulase, the support is constituted by wheat flour, the impregnating agent and the sticking agent are constituted by carboxymethyl cellulose (CMC).

31. A stable powder composition containing particles comprising:
a core containing at least one substance sensitive to temperatures of 20° C. to 100° C. and/or pressures of 10⁶ to 10⁷ Pa and/or a relative humidity of 60% to 100%, and at least one viscous compound the viscosity of which is greater than 5 Pa·s⁻¹ as impregnating agent for the sensitive substance; and
a coating for the core, which contains at least one viscous compound the viscosity of which is greater than 5 Pa·s⁻¹ as sticking agent, wherein the sensitive substance is not an enzyme intended for non-human animal food.

32. The stable powder composition according to claim 31, wherein the viscous compound is selected from the group consisting of vegetable gums, fermentation products, starchy bases, chitins and cellulose derivatives selected from the group consisting of carboxymethyl cellulose (CMC), methyl cellulose, ethyl cellulose, propyl cellulose, hydroxymethyl cellulose, hydroxyethyl cellulose, hydroxypropyl cellulose, hydroxypropyl methyl cellulose, ethyl hydroxyethyl cellulose, or microcrystalline cellulose.

33. The stable powder composition according to claim 31, wherein said sensitive substance is selected from the group consisting of proteins, vitamins, bacteria, yeasts, antioxidants, carotenoids, essential oils and pharmacologically active substances.

34. The stable powder composition according to claim 31, wherein the core contains at least one support of the sensitive substance.

35. The stable powder composition according to claim 34, wherein the support is selected from the group consisting of starch, flours, talc, beet pulp, maltodextrin, salts, corn distiller's grain.

36. The stable powder composition according to claim 34, wherein the impregnating agent and the sticking agent are identical.

37. The stable powder composition according to claim 36, wherein the impregnating agent and the sticking agent contain or are constituted by carboxymethyl cellulose (CMC).

38. The stable powder composition according to claim 34, wherein the sensitive substance represents 5 to 40% dry extract of the powder composition, the support represents 10 to 60% dry extract of the powder composition, the impregnating agent represents 5 to 40% dry extract of the powder composition and the sticking agent represents 5 to 30% dry extract of the powder composition.

39. A method for preparing a stable powder composition containing particles comprising:
a core containing at least one substance sensitive to temperatures of 20° C. to 100° C. and/or pressures of 10⁶ to 10⁷ Pa and/or a relative humidity of 60% to 100% and at least one viscous compound the viscosity of which is greater than 5 Pa·s⁻¹ as impregnating agent for the sensitive substance; and
a coating for the core, which contains at least one viscous compound the viscosity of which is greater than 5 Pa·s⁻¹ as sticking agent,
said method comprising:
a microgranulation stage for a mixture of said sensitive substance and said impregnating agent in order to obtain the core; and
a stage of coating said core with said sticking agent.

40. A method for preparing a stable powder composition containing particles comprising:
a core containing at least one substance sensitive to temperatures of 20° C. to 100° C. and/or pressures of 10⁶ to 10⁷ Pa and/or a relative humidity of 60% to 100%, at least one viscous compound the viscosity of which is greater than 5 Pa·s⁻¹ as impregnating agent for the sensitive substance and at least one support of the sensitive substance; and
a coating for the core, which contains at least one viscous compound the viscosity of which is greater than 5 Pa·s⁻¹ as sticking agent,
said method comprising:
a microgranulation stage for a mixture of said sensitive substance, said support and said impregnating agent in order to obtain the core; and
a stage of coating said core with said sticking agent.

41. The method according to claim 40, comprising a microgranulation stage for a mixture of said sensitive substance and said support, which have been co-dried, and all of the impregnating agent.

42. The method according to claim 40, comprising a microgranulation stage for a mixture of said sensitive substance, said support and all of the impregnating agent, which have been co-dried.

43. The method according to claim 40, comprising a microgranulation stage for a mixture of said sensitive substance, said support and some of the impregnating agent, which have been co-dried, and the remainder of the impregnating agent.

44. The method according to claim 40, comprising a co-drying stage for a mixture containing the sensitive substance, the support of the sensitive substance, and optionally all or some of the impregnating agent, the co-drying being carried out at an output air temperature of less than 60° C. and at a powder temperature of less than 45° C.

45. The method according to claim 40, comprising:
a co-drying stage for a mixture containing the sensitive substance and at least one support of the sensitive substance, the co-drying being carried out at an output air temperature of less than 60° C. and at a powder temperature of less than 45° C., in order to obtain an intermediate homogeneous powder; and
a microgranulation stage for the intermediate homogeneous powder with the impregnating agent in order to obtain a densified microgranulated powder constituted by uncoated particles.

46. The method of preparation according to claim 40, comprising:
a co-drying stage for a mixture containing the sensitive substance and at least one support of the sensitive substance, the co-drying being carried out at an output air temperature of less than 60° C. and at a powder temperature of less than 45° C., in order to obtain an intermediate homogeneous powder;
a microgranulation stage for the intermediate homogeneous powder with the impregnating agent in order to obtain a densified microgranulated powder constituted by uncoated particles corresponding to the abovementioned core; and
a stage of coating the uncoated particles of the densified microgranulated powder with the sticking agent in order to obtain a coated powder.

47. The method of preparation according to claim 40, comprising:
a co-drying stage for a mixture containing the sensitive substance and at least one support of the sensitive substance, the co-drying being carried out at an output air temperature of less than 60° C. and at a powder temperature of less than 45° C., in order to obtain an intermediate homogeneous powder;
a microgranulation stage for the intermediate homogeneous powder with the impregnating agent in order to obtain a densified microgranulated powder constituted by uncoated particles corresponding to the abovementioned core;
optionally a low-temperature drying stage for the densified microgranulated powder at a temperature of less than 45° C., in order to obtain a dried densified microgranulated powder;
optionally a sieving stage in order to obtain a sieved densified microgranulated powder;
a stage of coating the uncoated particles of the densified microgranulated powder, optionally dried and optionally sieved, with the sticking agent in order to obtain a coated powder; and
a drying stage for the coated powder in order to obtain a stable powder composition.

48. A pharmaceutical composition containing a stable powder composition containing particles comprising:
a core containing, as active substance, at least one substance sensitive to temperatures of 20° C. to 100° C. and/or pressures of 10⁶ to 10⁷ Pa and/or a relative humidity of 60% to 100% and at least one viscous compound the viscosity of which is greater than 5 Pa·s⁻¹ as impregnating agent for the sensitive substance; and
a coating for the core, which contains at least one viscous compound the viscosity of which is greater than 5 Pa·s⁻¹ as sticking agent, wherein the sensitive substance has at least 80% of its initial activity, in combination with a pharmaceutically acceptable vehicle.

49. A food composition comprising a stable powder composition according to claim 31, wherein the sensitive substance has at least 80% of its initial activity and is not an enzyme intended for non-human animal food, and at least one food product.

50. The food composition according to claim 49, wherein the composition is in the form of pellets or granules.
